(19) United States
(12) Patent Application Publication
(10) Pub. No.: US 2010/0292598 A1
(43) Pub. Date: Nov. 18, 2010

(54) Device for Monitoring Physical Fitness

(75) Inventors: Patricio Roschk, Kempten (DE); Ernst Merk, Weissenhorn (DE); Marco Bühler, Ulm (DE); Werner Meternek, Neu-Ulm (DE)

Correspondence Address: PAULEY PETERSEN & ERICKSON, 2800 WEST HIGGINS ROAD, SUITE 365, HOFFMAN ESTATES, IL, 60169, US

(73) Assignee: BEURER GMBH & CO. KG, Ulm (DE)

(21) Appl. No.: 12/223,038

(22) PCT Filed: Jan. 12, 2007
(86) PCT No.: PCT/EP2007/000247

§ 371 Date: Jul. 26, 2010

(30) Foreign Application Priority Data
Jan. 19, 2006 (DE) 10 2006 002 851.1

(51) Int. Cl. A61B 5/04

(52) U.S. Cl. 600/519

(57) ABSTRACT

The invention relates to a device for monitoring the physical fitness using a pulse watch part (1) for detecting heart pulse rate data and an evaluation apparatus for providing fitness information which can be displayed by means of a display device and for whose derivation a processing unit (16) is present which is designed for reading in and including personal supplementary data. The accuracy of the fitness information made available is favoured by the fact that the personal supplementary data comprises physiological additional data, for whose detection additional equipment with an additional measuring apparatus (2) is provided, which can be connected, in terms of data transfer, to the pulse watch part (1) directly or indirectly for the automatic transmission of the physiological additional data and/or heart pulse rate data.

### BACKGROUND OF THE INVENTION

1. Field of the Invention

This invention relates to a device for monitoring physical fitness equipped with a heart rate monitor component for detecting heart rate data and an evaluation device for providing fitness information that can be displayed by a display device and is derived by a processing unit, embodied for reading in and including supplementary personal data.

2. Discussion of Related Art

German Patent Reference DE 90 16 970 U1 discloses a device for monitoring physical fitness, equipped with a heart rate monitor for measuring heart rate, particularly during an athletic activity. With such heart rate monitors, a chest strap equipped with a pulse sensor is situated in the vicinity of the heart for recording the heart rate and converting the signals received in order to transmit them, preferably in telemetric fashion, to a receiver unit belonging to the heart rate monitor, which is similar to a wristwatch on the wrist of the user. The data received are evaluated in an evaluation device of the heart rate monitor, which has a processing unit, and are displayed by a display unit on the wrist component so that users can check their physical stress and fitness. The wrist component also has a memory device, which can be used to store heart rate data, for example also in connection with additional data such as the date and time, and can be used to record data that is adjusted by a manual input component or is supplied via a digital input component from a personal computer, which data can be incorporated into the data processing. Alternatively, this publication also teaches situating a processing unit not in a wrist component, but in a section of the chest strap of the heart rate monitor. Although it is possible in particular to obtain information about the training status via the connectable personal computer and to monitor fitness in a more detailed, even trend-based fashion, users frequently wish to obtain more information about their physical fitness in the simplest possible way.

In addition, with a heart rate monitor disclosed in European Patent Reference EP 0 117 330 A2, which has no wrist component, fitness parameters including a maximum oxygen consumption VO2max, among others, can be obtained and displayed based on corresponding data processing with a microprocessor.

Another heart rate monitor that has a data transfer interface for connection to a personal computer is disclosed in German Patent Reference DE 696 30 834 T2. Here, the personal computer is an additional apparatus suitable for providing more detailed evaluation, monitoring, and display of trends and it is also possible to transfer adjustment parameters to the heart rate monitor component, but this requires, among other things, a corresponding knowledge in the use of a personal computer and suitable programs.

German Patent Reference DE 103 52 188 A1 discloses a sensor apparatus for determining vital statistics of a person to be medically monitored, but this involves a medical monitoring by authorized, trained personnel.

### SUMMARY OF THE INVENTION

One object of this invention is to provide a device for monitoring physical fitness equipped with a heart rate monitor component, that makes it possible to obtain the most informative possible data about physical fitness with the least possible amount of effort.

This object is achieved with characteristics described in this specification and in the claims. The supplementary personal data include additional physiological data detected by an additional apparatus, having an additional measuring device that can be brought directly or indirectly into a data transfer connection with the heart rate monitor component for the automatic transmission of the additional physiological data and/or heart rate data.

The additional physiological data can be used to expand upon and refine the fitness information derived in the processing unit so that the users are given more precise information about their fitness state. The inclusion of data via the automatic data transfer connection occurs with no difficulty to the user and the evaluation is carried out with the inclusion of heart rate data. The data transfer connection can be produced by hard-wired or wireless transmission devices. For example, the heart rate monitor component is either a mobile unit carried by a user, such as worn on the wrist, or is a unit mounted to a piece of stationary training equipment, such as a treadmill or strength training machine.

In alternative embodiments of the device, the heart rate monitor component and the additional measuring device have matching interfaces. The processing component is either situated in the heart rate monitor component while the heart rate monitor component and the additional measuring device are embodied for the automatic transmission of the additional physiological data from the additional measuring device to the heart rate monitor component, or the processing component is situated in the additional measuring device while the heart rate monitor component and the additional measuring device are embodied for the automatic transmission of heart rate data from the heart rate monitor component to the additional measuring device.

In another embodiment, the heart rate monitor component and the additional measuring device have respective interface devices via which the heart rate data can be transmitted from the heart rate monitor component to the additional measuring device by a memory cache or the additional physiological data can be transmitted from the additional measuring device to the heart rate monitor component. The memory cache in this case can, for example, be a plug-in, easily transportable memory unit with a standardized interface or can also be contained in a computer.

In an embodiment for providing informative data about physical fitness, the additional measuring device is a scale and the additional physiological data are current weight data. This makes it possible for users to also obtain related information about their weight control and, for example, information about the influence of their dietary pattern in addition to obtaining information about their fitness state.

In an embodiment for providing more detailed information, the scale has an impedance measuring unit for determining the body fat percentage and the additional physiological data also advantageously include further information about the muscle mass percentage, body water percentage, and/or bone mass percentage.

Other evaluation, archiving, and monitoring possibilities are achieved when the additional apparatus has a computer that can be brought into a data exchange connection with the heart rate monitor.

Various possibilities for displaying the fitness information to the user are achieved if this information is shown on a display unit situated on the heart rate monitor, the scale, and/or the computer. The heart rate monitor thus offers one option of permitting detailed information to be read and/or heard even during the athletic activity, while the display unit on the scale can be larger and more complex in design in order, for example, to output more detailed graphs, tables, or spoken information.

Other additional data that influence physical fitness or the fitness state are generated when the measuring device is in a form of a diet scale. This also makes it possible to control physical fitness and/or weight, and weight can be controlled by derived weight data.

Including height, age, and gender in the personal supplementary data encourages a reliable evaluation. The supplementary data can, for example, be input manually, such as into the wrist unit, the scale, or the computer or can be determined and consulted automatically based on stored values.

A time sequence can be incorporated into the monitoring of physical fitness and into the related documentation because the time and date can be input into the heart rate monitor component, the additional measuring device, and/or the computer and can be included in the preparation of the fitness information by the processing unit.

If the processing unit includes past personal data and to establish trend analyses that can be displayed in graphical and/or tabular form by the display device, then users have clear information about the chronological development of their training state and physical fitness.

In order to plan the training and possibly also diet and to monitor the health status, it is useful to furnish additional data to the processing unit by providing the additional apparatus with a blood pressure measuring device, training devices, intelligent clothing, an accessory-equipped backpack that can be brought into a data transfer connection, a belt pouch, another portable container or the like, a portable phone that can be brought into a data transfer connection, and/or a temperature sensor device. For example, a temperature sensor device accommodated in a simple fashion in a chest strap can be used to monitor an overheating during the athletic activity.

In other advantageous embodiments for assessing fitness, the processing unit can calculate the maximum oxygen consumption VO2max, the basal metabolic rate BMR, and/or the active metabolic rate AMR.

An increased convenience of use is also achieved if the display unit has a warning display that emits a warning signal when the additional physiological data fall below or exceed a preset or presettable critical threshold of a measured or determined value.

### BRIEF DESCRIPTION OF THE DRAWINGS

This invention is explained in greater detail in view of exemplary embodiments with reference to the drawings, wherein:

FIG. 1 is a schematic block diagram of a device for monitoring physical fitness;

FIGS. 2 through 5 are schematic block diagrams of different embodiments of the device shown in FIG. 1, in various designs;

FIG. 6 shows various data groups for evaluation in the device;

FIG. 7 shows additional data groups for evaluation in the device; and

FIG. 8 is another depiction of data groups for evaluation in the device.

### DETAILED DESCRIPTION OF THE INVENTION

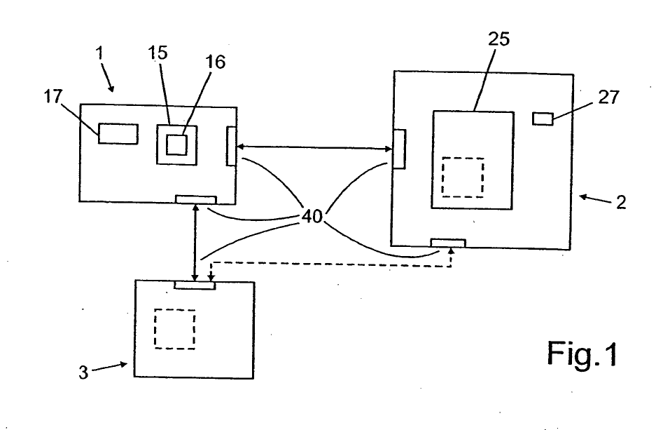

The essential components of a device for monitoring physical fitness shown in FIG. 1 are a heart rate monitor component 1, a scale component 2, and an additional component 3, in particular a computer. For example, the heart rate monitor component 1 can have a wrist unit with a receiver component for heartbeat signals and can have a chest strap with a pulse sensor that records these signals. In addition, a display unit 17 of a display device, such as an LCD and/or LED display unit or the like, an evaluation device 15, a processing device 16, and interfaces of an interface device 40 can be integrated into the heart rate monitor component 1. The interfaces can be embodied as wireless or hard-wired. With the interfaces, a data transfer can occur from and/or to the additional component 3 and/or from or to the scale component 2, with the scale component 2 and the additional component 3 having appropriately adapted interfaces. The data transfer between the heart rate monitor component 1 and the scale component 2 can also occur indirectly via the additional component 3, but a direct data transfer between the heart rate monitor component 1 and the scale component 2 is preferable. This combination of the heart rate monitor component 1 and scale component 2, with matching interfaces for an automatic data transfer, permits an advantageous combination of the heart rate monitor component 1 with a scale component 2 for determining fitness information by the evaluation device 15 and the processing device 16 because the processing, evaluation, and subsequent display of the data on the display unit can also include weight data and an associated control of the training results as well as a monitoring of the physical fitness state. The display unit 17 of the heart rate monitor component 1 is integrated into the wrist unit while an optional additional display unit 27 is integrated into the scale component 2 and can correspondingly be larger and more complex in design. The scale component 2 has a measuring device 25 for calculating the weight based on sensor data and, with a correspondingly more complex design, is equipped to calculate additional data such as body water percentage, muscle mass percentage, bone mass percentage, and/or body fat percentage, which are calculated based on an impedance measurement. Furthermore, a processing unit for deriving fitness information can additionally or alternatively be accommodated in the scale component 2, for example in connection with a measuring device 25.

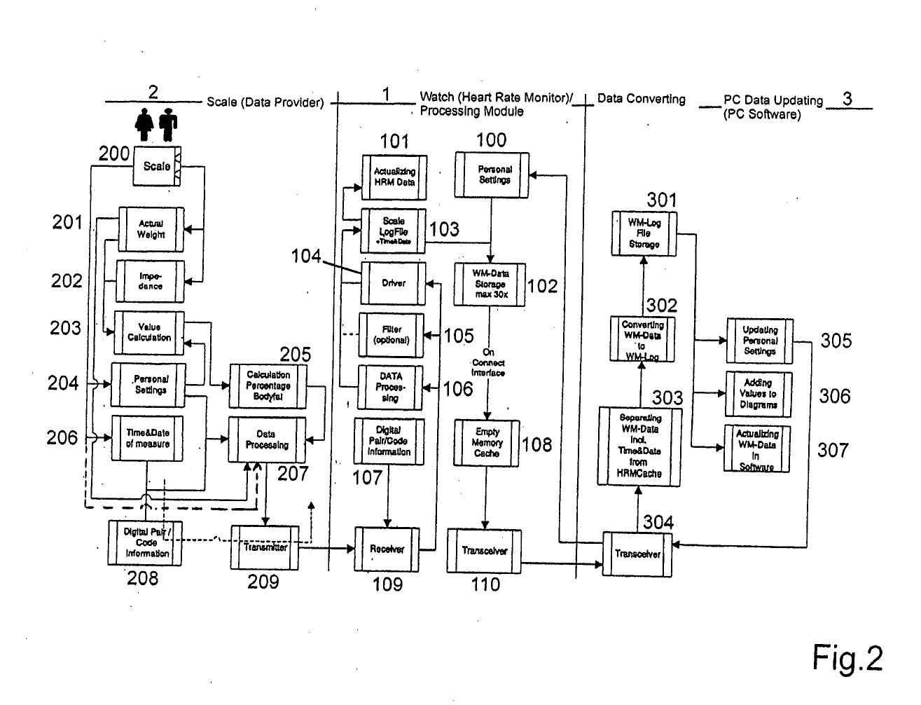

As shown in greater detail in FIG. 2, the heart rate monitor component 1 has a personal data setting unit 100 into which data relating to the user can be input manually or via an interface, such as using the additional component 3, in particular a computer. In addition, the updated heart rate data are received into a heart rate data updating unit 101 and can be associated with weight data and possibly the above-mentioned data including body fat percentage, body water percentage, muscle mass percentage, and/or bone mass percentage in a scale data memory unit 103 for additional physiological data. The additional physiological data generated by the scale component 2 are received into the heart rate monitor component 1 via a receiver component 109, in the process of which, the heart rate monitor component 1 can cooperate with an encoding/digitizing unit 107, and are furnished to the scale data memory unit 103 via a data processing unit 106, possibly a filter 105 for selecting essential data, and through the action of a driver 104. The additional physiological data obtained by the scale component 2 can be stored in this memory unit along with the date and time in a fashion similar to a file and can be removed from it in a corresponding fashion.

Furthermore, in the exemplary embodiment according to FIG. 2, the heart rate monitor component 1 contains a weight control memory unit 102 for storing a plurality of, for example up to 30 or 50, separate data items relating to weight control, thus making it possible to display a series of past weight control data and to review a change in body weight over time. The weight control data and other weight data can be supplied via a memory cache 108 to a transceiver component 110 of the heart rate monitor component 1 and supplied via it to another transceiver component 304 of the additional component 3 or computer. On the other hand, the additional transceiver component 304 can be used to supply the heart rate monitor component 1, for example the personal data setting unit 100, with data that are present or determined in the additional component 3. The data of the memory cache 108 can be supplied to the computer 3 as soon as the interface of the heart rate monitor component 1 is connected to the related interface of the computer 3 or a corresponding transmission command especially for this purpose has been entered. In addition, the memory cache 108 can also store a number of, such as 10 or 20, past heart rate data sets and training records that can be transmitted in the above-mentioned fashion to the additional component 3 or computer.

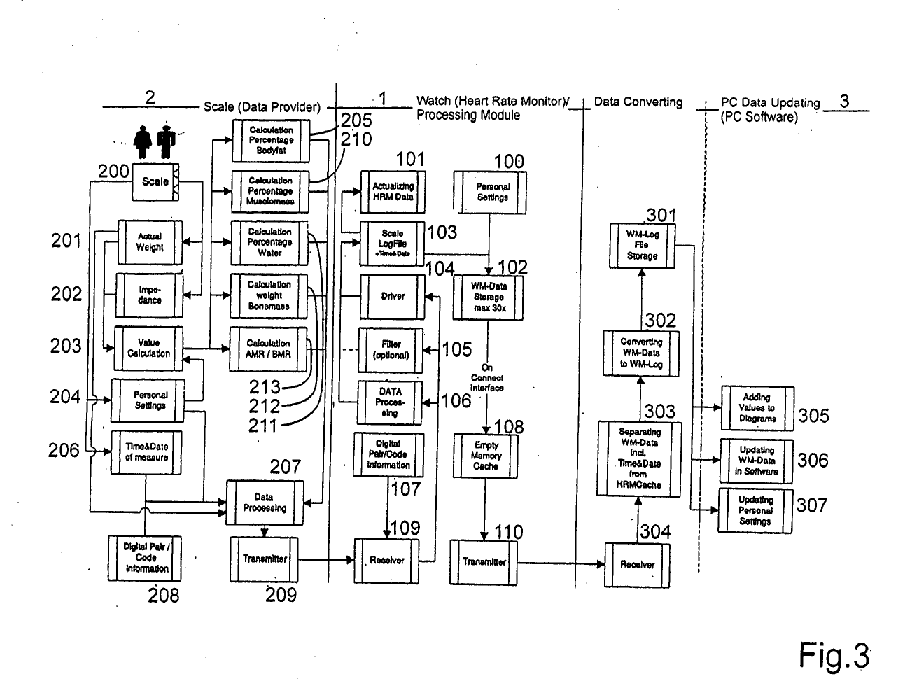

To collect weight data, the scale component 2 has a scale 200 with the above-mentioned measuring device 25 for measuring the current weight in the weight detection unit 201. The scale component 2 also has an impedance measuring device 202 that can, in a corresponding additional embodiment of the scale component 2, such as shown in FIGS. 3 through 5, be used to determine the above-mentioned additional physiological data such as the body water percentage, body fat percentage, muscle mass percentage, and bone mass percentage. In connection with personal data that have been input via a personal data input component 204, the corresponding additional physiological data are calculated in a value calculating component 203 and are then associated with a time and date that are generated, for example, in an optional measuring time and date unit 206 or alternatively in the heart rate monitor component 1, for a subsequent, uniquely defined evaluation. In a body fat calculation component 205, data relating to the body fat percentage are calculated and, together with the weight data, can be further processed in a data processing component 207, including the time and date. To control the individual units, they can be connected to an encoding/digitizing component 208. The data of the scale component 2 are transmitted via a transmitter component 209 to the receiver component 109 of the heart rate monitor component 1.

The additional component 3, which can at least partially include a computer and, via the transceiver component 304, receives the heart rate data, which are associated with the additional physiological data including the weight data, and optionally, data that have already been generated based on them, has a separating component 303 as an additional component, which is connected to the additional transceiver component 304 and is for separating the supplied data, for example into weight data and heart rate data or similarly suitable subdivided data for the training state on the one hand and weight control on the other. This separation then makes it possible for a weight data converting component 302 and a weight data memory unit 301 to carry out a corresponding, separate consideration in connection with weight control. In another region of the additional component 3, which can be accommodated in the computer, it is then possible to carry out further evaluations in an updating component 305 of the personal data, a graph supplementing component 306, and/or an updating component 307 for the weight data. It is also possible for the resulting data to be supplied back to the heart rate monitor component 1, for example the personal data setting unit 100, in order to store user-specific information there, to make them displayable as needed, and to prepare them as output values for a subsequent training cycle.

The above-mentioned components 301 through 307, in particular the components 301 through 303 of the additional component 3 can be alternatively or partially integrated into the heart rate monitor component 1 and/or the scale component 2. To the extent that they relate to data processing, evaluation, and control, the components can be embodied as program components of one or more microprocessors or microcontrollers.

By contrast, with the exemplary embodiment of the device for monitoring physical fitness shown in FIG. 2, in the embodiment shown in FIG. 3, the scale component 2 is added to, namely with a muscle mass calculation component 210, a body water percentage calculation component 211, a bone mass percentage calculation component 212, and a metabolism calculation component 213 for calculating the basal metabolic rate BMR and the active metabolic rate AMR. The additional physiological data derived from these, such as the body fat percentage, muscle mass percentage, body water percentage, and bone mass percentage, can be used to obtain additional information about the influence of the training and possibly an altered diet for weight control as well as fitness information. In addition, this provides important assessment criteria for the monitoring and control of the resulting basal metabolic rate and active metabolic rate. Another embodiment relates to the transceiver component 110 and the additional transceiver component 304 being respectively embodied, one as a transmitter component and the other as a receiver component if it is desirable to have a data transfer in only one direction.

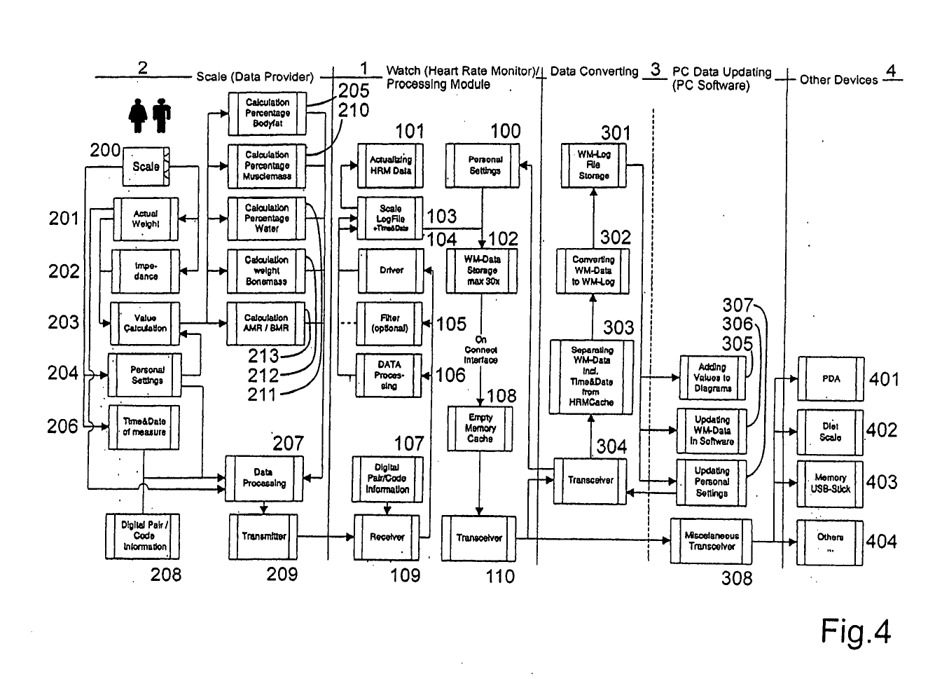

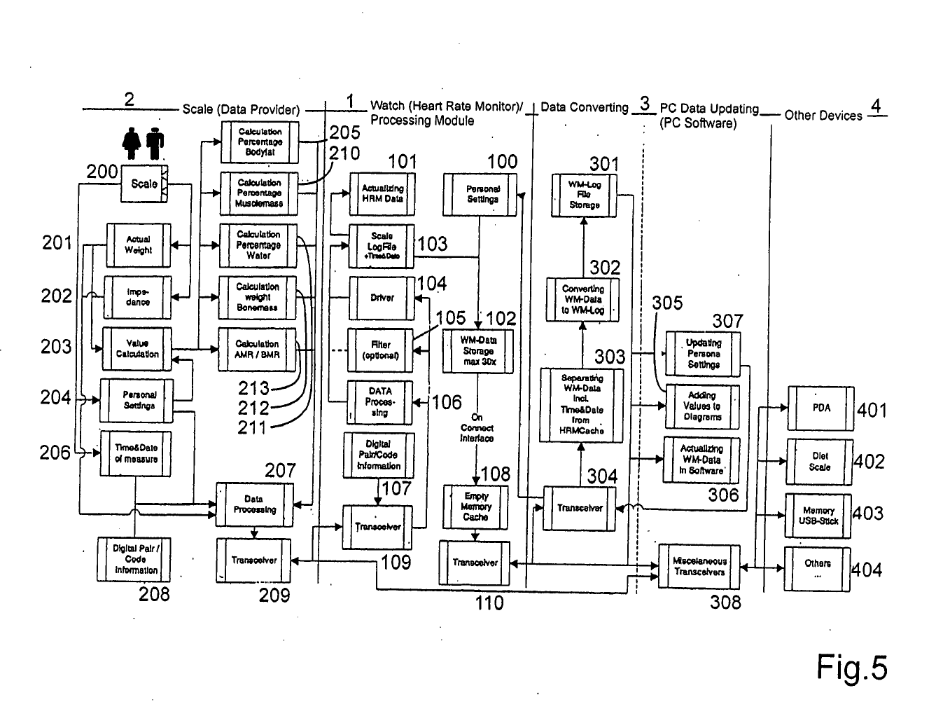

In the embodiment shown in FIG. 4, other additional accessories 4 are indicated as part of the additional apparatus, such as a minicomputer PDA (personal digital assistant) 401, a diet scale 402, a memory unit 403 for being plugged such as into a USB interface, or other accessories 404 such as a blood-pressure measuring device, a temperature sensor that can be integrated into the chest strap of the heart rate monitor component 1, various training devices such as ergometers, fans, motors or the like, intelligent clothing, an intelligent backpack, or a mobile telephone with corresponding interfaces. The drawing also shows transceiver components 308 suitable for them. According to FIG. 5, a two-way connection is shown between the additional component 3, in particular a computer, and the scale component 2, which components have corresponding transceiver components 209 and 308 and interfaces.

Possibilities for the transmission of data between the heart rate monitor component 1, the scale component 2, the optionally provided additional component 3, in particular a computer, and possibly additional accessories of the additional apparatus include various possibilities, for example a wireless transmission in the microwave range, such as a few GHz, a USB interface, an IR transmission, a Bluetooth transmission device, such as in the microwave range, a WLAN transmission, an exchange by memory cards or a plug-in memory unit, a suitable chip, a transmission using transponder technology, or another suitable magnetic, optical, or acoustic, such as ultrasonic, transmission method. These also include various combinations between the various devices, even ones known by other designations.

With separation of the detected or evaluated data into those relating to weight control and those relating to training progress, it is possible for separate evaluations to be carried out in respectively associated files relating to respective users on the basis of personal data that has been acquired. The time and date are each advantageously displayed along with the respective data and associated with them for analysis purposes. This step makes it possible for tabular overviews or graphs to be automatically generated, displayed, and if so desired, also printed and/or transmitted to other devices. The training records and weight control data can be used to establish trend analyses. The calculations and evaluations are carried out in the heart rate monitor component 1, the scale component 2, and/or the additional component 3 by software and corresponding programming.

With the weight control data from the scale 200, the individual values are updated, for example by software, in the evaluation unit 15 of the heart rate monitor component 1 and/or of the scale component 2 or additional component 3, in particular the computer so that in addition, the graphs or tabular depictions based on them are updated, preferably by software. In addition, the heart rate monitor component 1 and the scale component 2 can transmit the personal data to the computer, thus assuring that the computer always has the most up-to-date values corresponding to those in the heart rate monitor component 1 and the scale unit 2. It is possible to use the computer to update the personal data of the heart rate monitor component 1 and scale component 2, thus facilitating input and adjustment.

The processing unit 16, which is preferably situated in the evaluation device 15 of the heart rate monitor component 1, but can also be situated at least partially in the scale component 2 or the additional component 3, in particular the computer, is embodied for determining fitness information that can be displayed via the display unit. The additional physiological data, in particular the measured weight data or values derived from it, are included in the fitness information as explained above. A significant measure for physical fitness is the oxygen volume consumed during maximum performance. This VO2max value represents the maximum quantity of oxygen that can be consumed in one minute per kilogram of body weight. People who are more physically fit can train more intensively than those who are not in such good physical condition. Various studies show that the VO2max value can be increased if the training takes place in a range between 65% and 85% of the maximum heart rate. A training of this kind must take place for at least 20 minutes three to five times per week. On average, the value for male athletes is 3.5 liters per minute and for female athletes is 2.7 liters per minute.

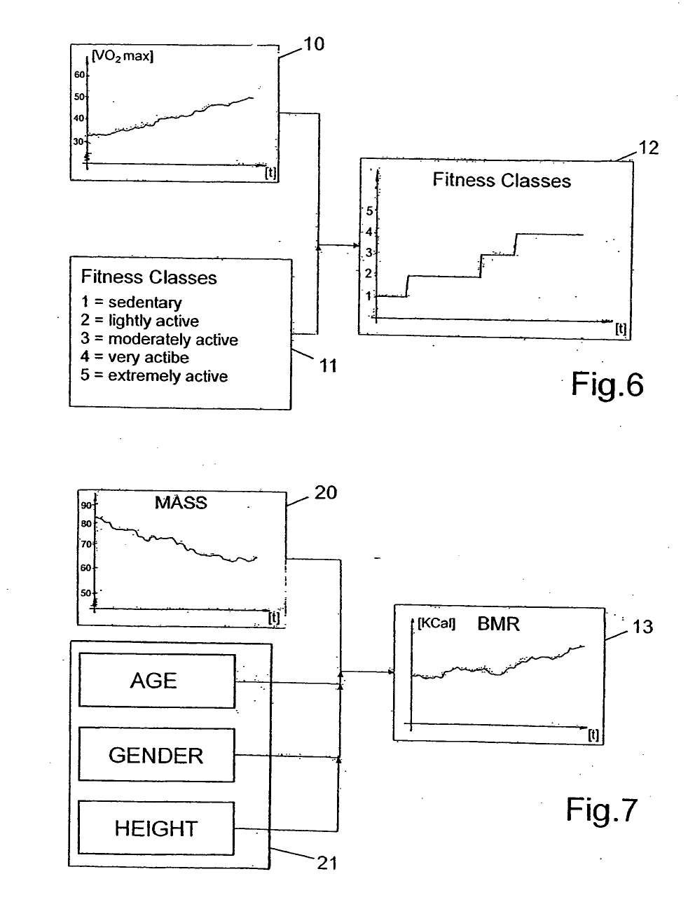

The active metabolic rate AMR is an additional indicator of physical fitness. The active metabolic rate represents the personal caloric expenditure, which is based on the personal athleticism and the personal basal metabolic rate BMR. The results of a plurality of past data acquisitions are consulted in order to determine the active metabolic rate and basal metabolic rate. In the first step, the personal basal metabolic rate and a fitness class are determined based on a fitness test carried out in accordance with a certain standardized scheme. This process is shown in FIGS. 6, 7, and 8. First, the values for the oxygen consumption volume 10 VO2max are determined on the basis of a known mathematical relationship that incorporates weight, age, gender, time required to travel a fixed distance, and heart rate at the end of the test.

The basal metabolic rate BMR is also determined on the basis of a known mathematical relationship that incorporates height, weight, age, and gender.

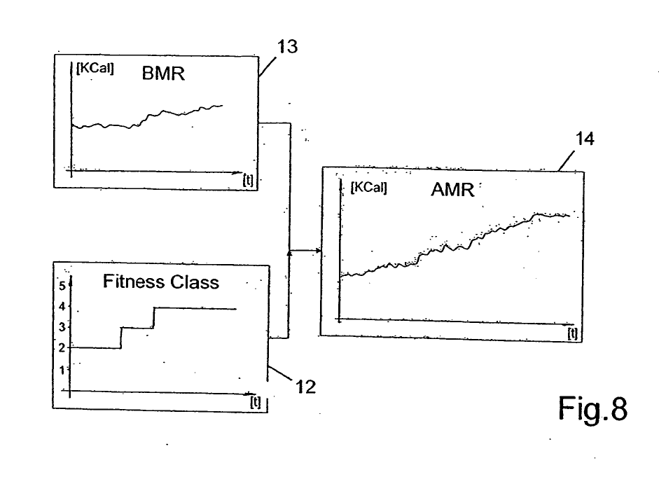

The determined basal metabolic rate BMR 13 and fitness class are then used to determine the active metabolic rate AMR 14, likewise on the basis of a known mathematical relationship, as shown in FIG. 8. These values are used to obtain significant elements of fitness information.

In addition, a weight control can be carried out with the device used. This can be carried out in the scale component 2, the heart rate monitor component 1, or the additional component 3, in particular a computer, on the basis of the data obtained. For example, this function can be used to reduce the body weight in a time span previously input by the user while also permitting precise monitoring of the training in order to attain this goal. A suggested training regimen can be established based on the current weight, the desired weight, and the time span. With the information obtained during training, it is also possible to record the weight curve, to ascertain the trend, to pursue goals, and if need be, to adapt the training and/or diet. With the software, it is possible to input the diet quality. This can be subjectively divided, for example, into three categories as a daily value. On the basis of this, the software can generate a graph that qualitatively represents the diet.

It is possible to calculate a caloric expenditure of the training. In order to establish a training program based on the additional caloric expenditure determined with the weight control, the estimated caloric expenditure can be displayed for each planned training unit. The calorie calculation of the weight check informs users of their weekly target value for the additional caloric expenditure. After the planned training units per week are input, the actual caloric expenditure per training unit and per week is displayed. It is thus possible to estimate how much training is required in order to achieve the established weight goal. In order to calculate the caloric expenditure per training unit, an activity parameter associated with the respective sport type is established and the calculated or input average heart rate is included.

Weight reduction forms another function. To use this function, users input the weight in kilograms by which their body weight should be reduced as well as the corresponding span of time scheduled. A safety questionnaire prevents the setting of impossible goals. The weight reduction per day is calculated based on the weight difference and the number of days and then a monitoring is carried out over time, including the inputting of corrections as needed.

In connection with weight reduction and athletic training, it is also possible to carry out a calorie calculation. To do so, the diet, the basal metabolic rate BMR, and the weight difference per day are used to calculate the additional caloric expenditure required. The additional caloric expenditure due to athletic activity can be influenced by manipulating the quantity of calories in the energy supply. This predetermines a mathematical relationship for the calculation. It is also possible to input training data manually.

In order to obtain further data as additional physiological data, the device can also detect the body temperature and/or body moisture due to perspiration. The temperature sensor for temperature detection here is advantageously accommodated in a temperature chest strap that can be combined with the strap of the heart rate monitor 1. It is thus possible during training to also monitor target values as a function of the detected body temperature and to announce them via the display unit when they are exceeded.

It is also advantageous for the heart rate monitor component 1, the scale component 2, the additional component 3, and optionally the additional devices to be embodied for calculating in various units of measure, such as based on the metric, inch, imperial, or UK system.

It is also advantageous for the display device, for example in the heart rate monitor component 1, the additional measuring device 2, and/or the additional component 3, to have a warning display that emits a warning signal when the additional physiological data fall below or exceed a preset or presettable critical threshold of a measured or determined value. For example, this can be used to notify training athletes when their body water percentage falls below a critical value.

1. A device for monitoring physical fitness having a heart rate monitor component (1) for detecting heart rate data and an evaluation device for providing fitness information that can be displayed by a display device and is derived by a processing unit (16) for reading in and including supplemental personal data, the device comprising:
the supplementary personal data including additional physiological data detected by an additional apparatus having an additional measuring device (2) and can be brought directly or indirectly into a data transfer connection with the heart rate monitor component (1) for an automatic transmission of the additional physiological data and/or the heart rate data.

2. The device as recited in claim 1, wherein the heart rate monitor component (1) and the additional measuring device (2) have matching interfaces, the processing component is situated in the heart rate monitor component (1) while the heart rate monitor component (1) and the additional measuring device (2) are embodied for the automatic transmission of the additional physiological data from the additional measuring device (2) to the heart rate monitor component (1), or the processing component is situated in the additional measuring device (2) while the heart rate monitor component (1) and the additional measuring device (2) are for an automatic transmission of heart rate data from the heart rate monitor component (1) to the additional measuring device (2).

3. The device as recited in claim 2, wherein the heart rate monitor component (1) and the additional measuring device (2) have respective interface devices via which the heart rate data can be transmitted from the heart rate monitor component (1) to the additional measuring device (2) by a memory cache or the additional physiological data can be transmitted from the additional measuring device (2) to the heart rate monitor component (1).

4. The device as recited in claim 3, wherein the additional measuring device (2) is a scale and the additional physiological data are current weight data.

5. The device as recited in claim 4, wherein the scale has an impedance measuring unit for determining a body fat percentage and information about the body fat percentage is included in the additional physiological data.

6. The device as recited in claim 5, wherein the additional physiological data also include information regarding a muscle mass percentage, a body water percentage, and/or a bone mass percentage.

7. The device as recited in claim 6, wherein the additional apparatus has a computer (3) that can be brought into a data exchange with the heart rate monitor.

8. The device as recited in claim 7, wherein the fitness information can be displayed on a display unit situated on the heart rate monitor (1), the scale (2) and/or the computer (3).

9. The device as recited in claim 8, wherein the additional measuring device (2) is a diet scale.

10. The device as recited in claim 9 wherein the supplemental personal data include a height, an age, and a gender.

11. The device as recited in claim 10 wherein a time and date can be input into the heart rate monitor component (1), the additional measuring device (2) and/or the computer (3) and can be included in a preparation of the fitness information by the processing unit.

12. The device as recited in claim 11, wherein the processing unit includes past personal data and establishes trend analyses that that can be displayed in graphic and/or tabular form by the display unit.

13. The device as recited in claim 12 wherein for furnishing additional data to the processing unit, the additional apparatus has a blood-pressure measuring device, training devices, an intelligent clothing, an accessory-equipped backpack that can be brought into a data transfer connection, a belt pack, a portable container, a portable phone that can be brought into a data transfer connection and/or a temperature sensor device.

14. The device as recited in claim 13 wherein the data processing unit calculates the maximum oxygen consumption VO2max, the basal metabolic rate BMR, and/or the active metabolic rate AMR.

15. The device as recited in claim 14 wherein the display device has a warning display that emits a warning signal when the additional physiological data fall below or exceed a preset critical threshold or a presettable critical threshold of a measured value or a determined value.

16. The device as recited in claim 1, wherein the heart rate monitor component (1) and the additional measuring device (2) have respective interface devices via which the heart rate data can be transmitted from the heart rate monitor component (1) to the additional measuring device (2) by a memory cache or the additional physiological data can be transmitted from the additional measuring device (2) to the heart rate monitor component (1).

17. The device as recited in claim 1, wherein the additional measuring device (2) is a scale and the additional physiological data are current weight data.

18. The device as recited in claim 17, wherein the scale has an impedance measuring unit for determining a body fat percentage and information about the body fat percentage is included in the additional physiological data.

19. The device as recited in claim 4, wherein the additional physiological data include information regarding a muscle mass percentage, a body water percentage, and/or a bone mass percentage.

20. The device as recited in claim 1, wherein the additional apparatus has a computer (3) that can be brought into a data exchange with the heart rate monitor.

21. The device as recited in claim 1, wherein the fitness information can be displayed on a display unit situated on the heart rate monitor (1), the scale (2) and/or the computer (3).

22. The device as recited in claim 1, wherein the additional measuring device (2) is a diet scale.

23. The device as recited in claim 1, wherein the supplemental personal data include a height, an age, and a gender.

24. The device as recited in claim 1, wherein a time and date can be input into the heart rate monitor component (1), the additional measuring device (2) and/or the computer (3) and can be included in a preparation of the fitness information by the processing unit.

25. The device as recited in claim 1, wherein the processing unit includes past personal data and establishes trend analyses that can be displayed in graphic and/or tabular form by the display unit.

26. The device as recited in claim 1, wherein for furnishing additional data to the processing unit, the additional apparatus has a blood-pressure measuring device, training devices, an intelligent clothing, an accessory-equipped backpack that can be brought into a data transfer connection, a belt pack, a portable container, a portable phone that can be brought into a data transfer connection and/or a temperature sensor device.

27. The device as recited in claim 1, wherein the data processing unit calculates a maximum oxygen consumption VO2max, a basal metabolic rate BMR, and/or an active metabolic rate AMR.

28. The device as recited in claim 1, wherein the display device has a warning display that emits a warning signal when the additional physiological data fall below or exceed a preset critical threshold or a presettable critical threshold of a measured value or a determined value.
